(19) United States
(12) Patent Application Publication
(10) Pub. No.: US 2007/0082390 A1
(43) Pub. Date: Apr. 12, 2007

(54) Scaffold Carrier Cartridge

(76) Inventors: Abel Z. Hastings, Durham, NC (US); Neil Robbins, Cary, NC (US)

Correspondence Address: DAVID W. HIGHET;BECTON, DICKINSON AND COMPANY, 1 BECTON DRIVE, MC110, FRANKLIN LAKES, NJ, 07417, US

(21) Appl. No.: 11/532,688

(22) Filed: Sep. 18, 2006

Related U.S. Application Data
(60) Provisional application No. 60/717,187, filed on Sep. 16, 2005.

(51) Int. Cl. C12M 1/22; C12M 1/34

(52) U.S. Cl. 435/305.2; 435/297.5; 435/288.4; 435/299.1

(57) ABSTRACT

A scaffold handling system comprising a multi-well carrier including an array of well units wherein, in each well unit, an independent scaffold or tissue slice may be held and a biological experiment may be performed. The carrier may include any number of well units. In operation, the scaffold handling system is configured and dimensioned to mate with a multi-well plate.

### CROSS-REFERENCE TO RELATED APPLICATIONS

The present application claims priority to U.S. Provisional Patent Application Ser. No. 60/717,187 filed Sep. 16, 2005, the entire contents of which are incorporated herein by reference.

### FIELD OF THE INVENTION

The present invention relates generally to an apparatus for cell and tissue culture, and, more particularly, to a system and method for manipulating or handling scaffolds and tissue slices in a platform for high throughput and parallel screening, as well as tissue engineering.

### BACKGROUND OF THE INVENTION

Tissue engineering is a strategy for regenerating natural tissue. Cell culture in the context of tissue engineering often requires a three-dimensional scaffold for cell support. A scaffold having a three-dimensional porous structure is a prerequisite in many tissue culture applications, such as chondrocyte and hepatocyte cell culture, because these cells would otherwise lose their cellular morphology and phenotype expression in a two-dimensional monolayer cell culture. For regenerating natural tissue, the quality of the three-dimensional matrix can greatly affect cell adhesion and growth, and determine the success of tissue regeneration or synthesis. An optimal matrix material would promote cell binding, cell proliferation, expression of cell-specific phenotypes, and the activity of the cells.

In practical use, however, scaffolds can prove to be challenging to incorporate into standard biology. Scaffolds are inherently low density, this means that scientists performing biological experiments utilizing scaffolds must be careful while handling them in order to minimize the chances of damaging the scaffold. For example, when the scaffolds are handled within a biological safety hood they may be displaced by the airflow in the hood. Furthermore, scaffolds typically are sensitive to static electricity further complicating handling and manipulation. In addition, due to their relatively small size, usually about 30 mm³, a typical scaffold is handled with the aid of a tweezer or other similar instrument which can be cumbersome, frustrating and/or inefficient for high throughput experimentation. As a result, heretofore the use of scaffolds has been technique intensive and experimentation data has been viewed with some degree of skepticism due to the challenge and variability of handling them.

A need exists for a system and method for manipulating scaffolds for use in biological experiments. In particular, a need exists for a device that addresses the aforementioned handling challenges by containing and positioning scaffolds for easy use and permits accurate movement and positioning in a repeatable fashion. Furthermore, a need exists for a device for handling scaffolds for use in high throughput or multiple parallel biological experiments.

### SUMMARY OF THE INVENTION

The present invention is directed to a scaffold handling system comprising a multi-well carrier including an array of well units wherein, in each well unit, an independent scaffold may be held and a biological experiment may be performed. The carrier may include any number of well units. In operation, the scaffold handling system is configured and dimensioned to mate with a multi-well plate.

### BRIEF DESCRIPTION OF THE DRAWINGS

The invention will be more readily understood with reference to the embodiments thereof illustrated in the attached drawing figures, in which:

FIG. 1 is a side view of one embodiment of a system according to the present invention;

FIG. 2 is a perspective view of one embodiment of a carrier of the system of FIG. 1;

FIGS. 3 and 3A are partial perspective views of alternate embodiments the carrier of FIG. 2 depicting a single well as seen from the bottom;

FIGS. 4 and 4A are partial perspective views of alternate embodiments of the carrier of FIG. 2 depicting a single well as seen from the top;

FIGS. 5, 5A and 6, 6A are partial side views of alternate embodiment of a single well shown without and with a screen;

FIG. 7 is a perspective view of one embodiment of an adapter according to the invention;

FIGS. 8-9 are bottom and side views, respectively, of the adapter of FIG. 7; and

FIGS. 10-11 are exploded views of an assembly of the adapter of FIG. 7 with the carrier of the system of FIG. 1.

### DESCRIPTION OF THE PREFERRED EMBODIMENTS

The present invention relates to handling a cell adherent structure, and, more particularly, to a system and method for manipulating or handling scaffolds in a platform for performing biological experiments in a high throughput and/or parallel screening environment.

As referred to in the multiple embodiments, the cell adherent structure is a three-dimensional scaffold, such as a porous body having a plurality of three-dimensional cell adherent surfaces, however, in alternate embodiments, the cell adherent structure may be two-dimensional, such as a slide or plate having a two-dimensional cell adherent surface. In other alternate embodiments, the cell adherent structure may have varied shapes such as, for example, a tubular or cylindrical shape, such that a transplantable medical device/implant with a biological component may be engineered in a high throughput device. In this regard, cells and/or tissue may adhere or grow upon the tubular structure to grow cell or tissue containing tubes such as, for example, vascular grafts, stents, neural tubes, shunts, etc., for transplantation into the body of a patient. In other embodiments, cartilage and/or bone may be grown or engineered in a predetermined shape.

The scaffolds can be made from any type of polymer, ceramic, metal or mixture of any type suitable for adhering cells thereto. In a preferred embodiment, the scaffold is made from a hydrogel-based material, which may be synthesized from covalently crosslinked alginate, hyalrunic acid or a blend of the two polysaccharides at any mixing percentage as desired. For example, the mixing percentage may be tailored to achieve a desired degradation profile for the final application. In alternate embodiments, the scaffolds may be made of other suitable materials, such as those disclosed in U.S. Patent Publication No. 2004/0147016 entitled “Programmable scaffold and methods for making and using same”, the entire contents of which are incorporated by reference. In one preferred embodiment, the scaffold may be a porous structure having randomly aligned pores. In alternative embodiments, scaffolds may be used that have directionally aligned pores such that a less random pore pattern may be attained and fluid flow may be further assured of navigating or flowing through all of the pores of the scaffold. In alternate embodiments, the scaffolds may be modified with any number or type of cell signaling or cell interacting molecule, such as those disclosed in U.S. Patent Publication No. 2004/0147016, entitled “Programmable scaffold and methods for making and using same, ” the entire contents of which are incorporated by reference.

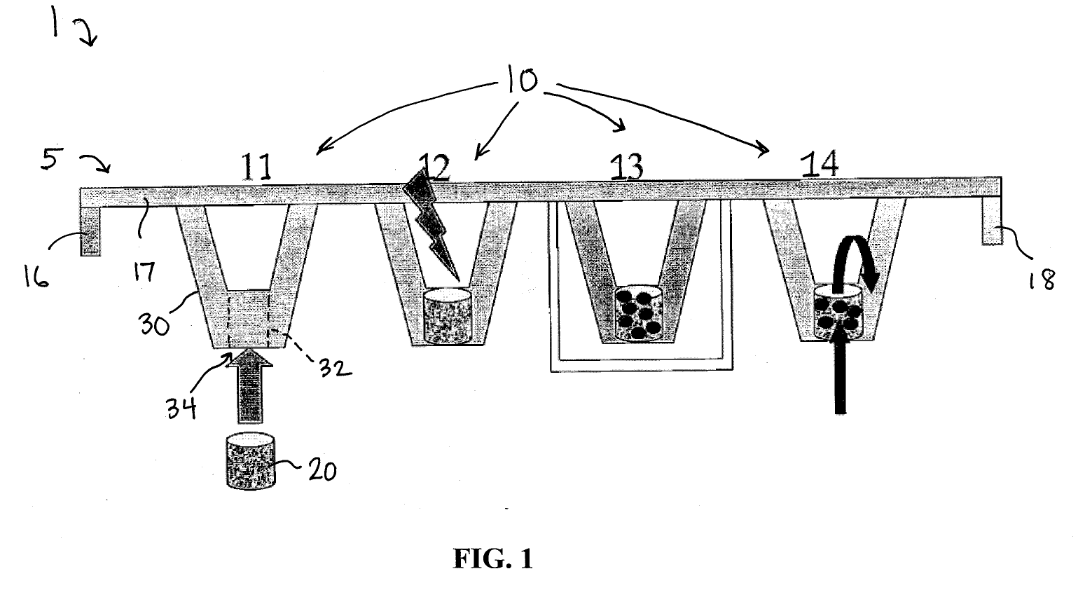

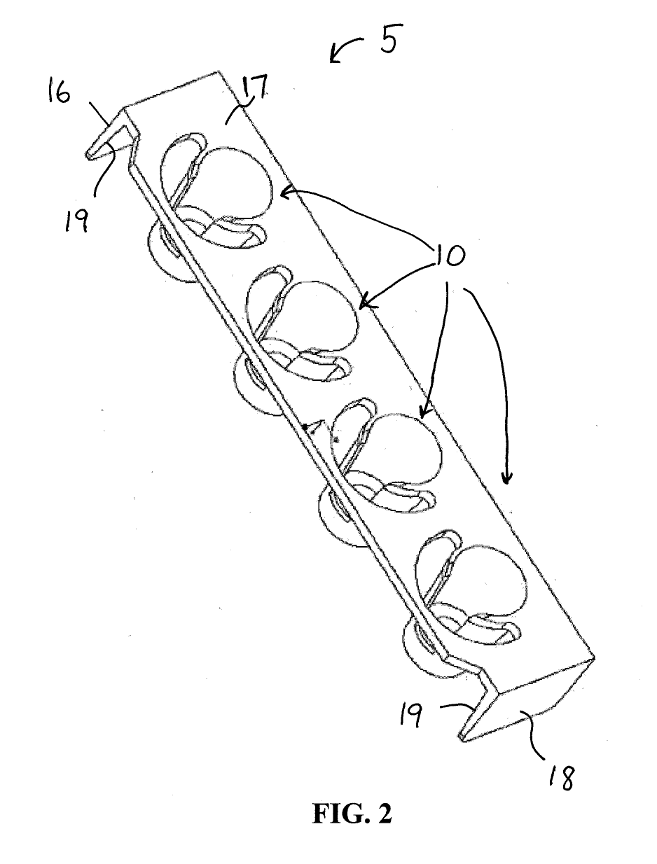

Referring now to FIGS. 1 and 2, a preferred embodiment of a scaffold handling system 1 generally includes a multi-well cartridge or carrier 5 comprising an array of well units 10 wherein, in each well unit, an independent scaffold 20 may be held and a biological experiment may be performed. As shown in FIG. 1, in one embodiment, carrier 5 of scaffold handling system 1 comprises four well units 11, 12, 13, and 14 and includes sidewalls or flanges 16 and 18 extending distally from the lateral ends of cross-member 17 to mate with a multi-well plate. In alternate embodiments, however, any number or multiple of well units 10 may be included in carrier 5. For example, in one variation carrier 5 may have one well unit. In another exemplary embodiment, carrier 5 may have 8 well units. In yet another embodiment, carrier 5 may have 3 well units.

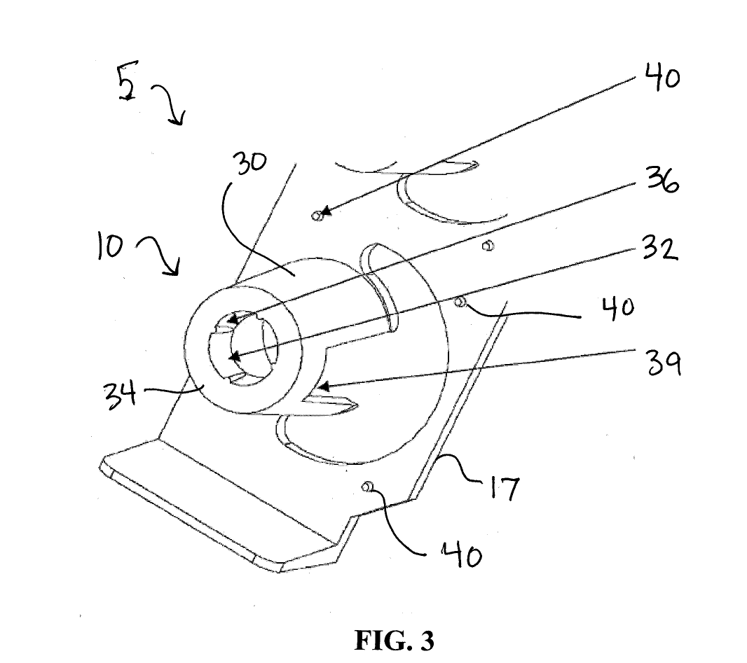

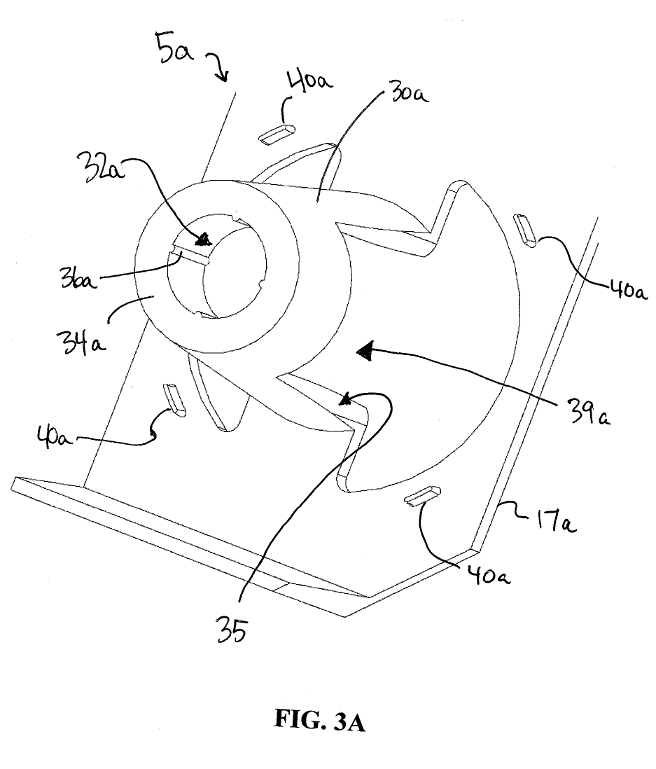

Each well unit 10 generally comprises a frustoconical or tapered body 30 extending distally from the top of carrier 5 and includes a scaffold holding chamber 32 at the distal end 34. A cell adherent structure or scaffold 20 is preferably housed or held within each well unit 10 to facilitate high density cell culture growth. In one embodiment, as illustrated by the upward arrow in the left-most well unit 11 in FIG. 1, the cell adherent structure is coupled or loaded into to the well unit 10 about a distal (bottom) end 34; however, in alternative exemplary embodiments (e.g. FIGS. 3A, 4A, 5A, and 6A) the cell adherent structure may be coupled or loaded into the well unit from the top or about the proximal end. For example, a three-dimensional scaffold 20 may be coupled, molded, bonded, synthesized, or otherwise attached to the distal chamber 32. In one preferred embodiment, scaffold 20 may be releasably plugged into or attached to chamber 32 for example by friction fit.

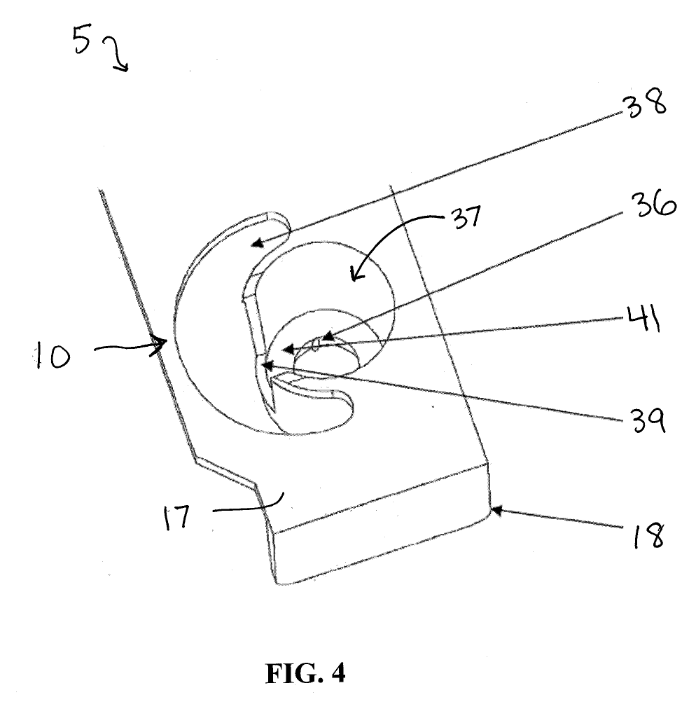

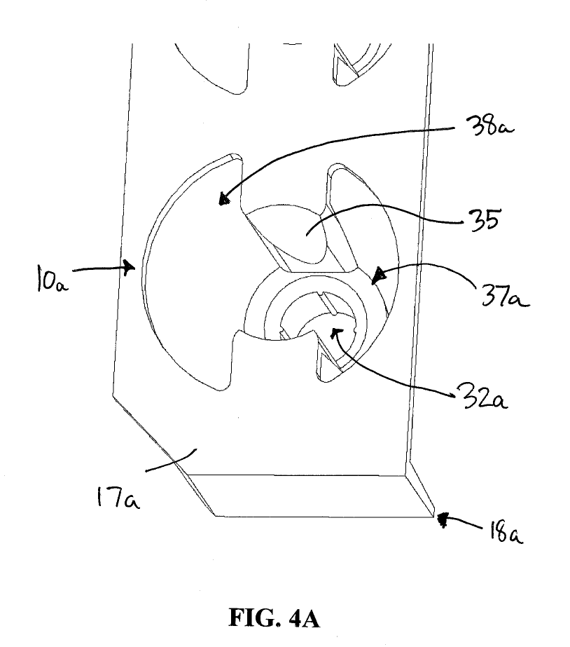

Referring to FIG. 3, a bottom perspective view of carrier 5 is shown depicting one of the well units 10. In one embodiment, scaffold holding chamber 32 is tapered, i.e. wider at the distal end of the well unit and narrower at the top or proximal end of the chamber. This tapered feature of chamber 32 may accommodate a range of scaffold sizes. For example, in one embodiment, chamber 32 may accommodate scaffolds with diameters ranging from about 4.8 mm to about 5.1 mm. In addition, one or more nubs or protrusions 36 may extend radially inward from the perimeter of chamber 32 to further grip or hold a scaffold therein by friction. In an alternate embodiment, shown in FIG. 3A, scaffold holding chamber 32a may be tapered in the opposite direction, i.e. wider at the top or proximal end of the chamber and narrower at the distal end of the well unit. In this regard, in the embodiment of FIG. 3A, the cell adherent structure may be coupled or loaded into the well unit from the top or about the proximal end. In one embodiment, a scalloped region 35, best seen in FIG. 4A, may be provided along the interior of well unit 10a to facilitate insertion of the cell adherent structure from the top. In some embodiments, chambers 32, 32a may be tapered between about 1 degree and 6 degrees along its length.

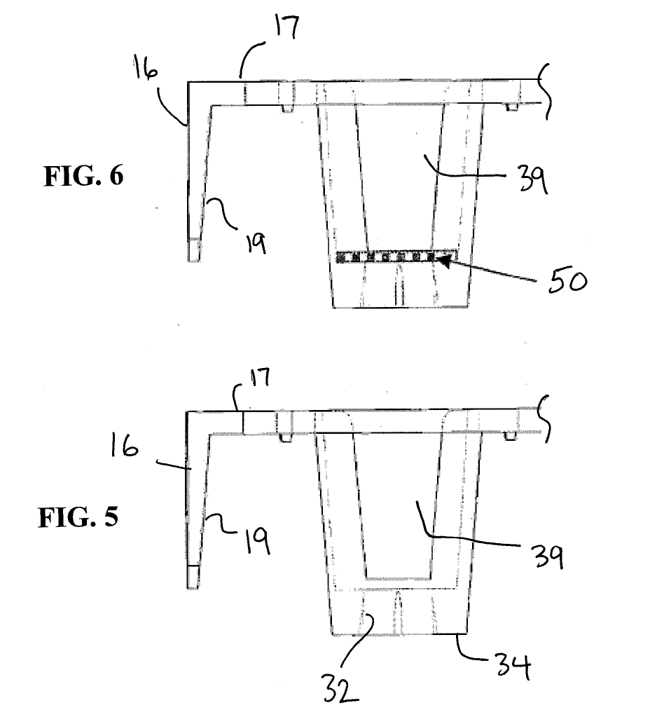

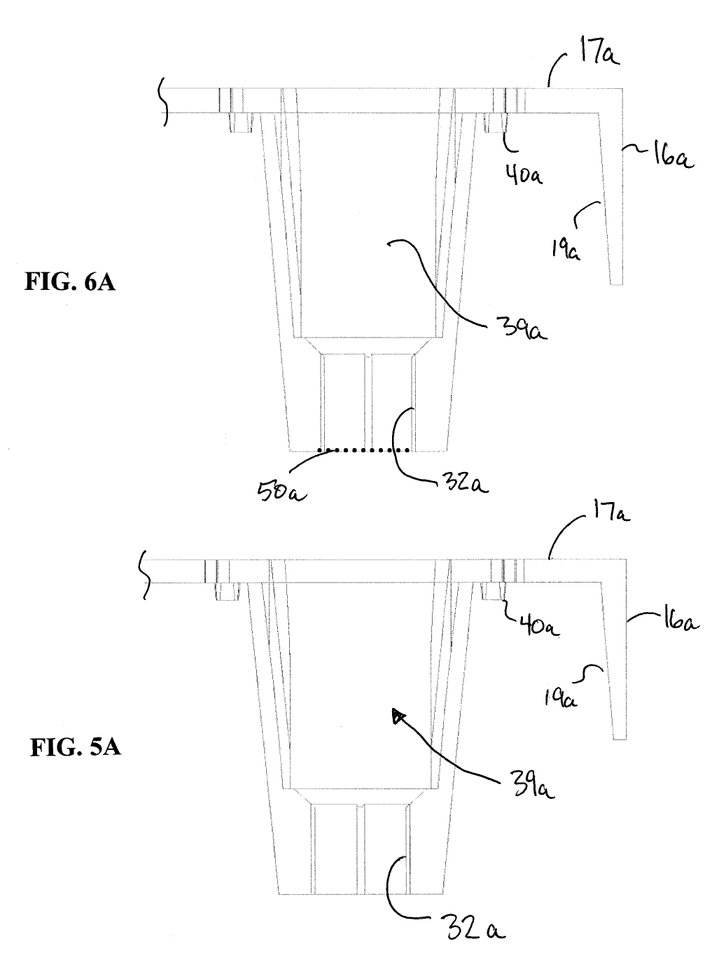

Referring to FIG. 4, a top perspective view of carrier 5 is shown depicting one of the well units 10. The top or proximal end of each well unit 10 defines an opening 37 to permit physical and visual access to a scaffold 20 held therein. In addition, a window 38 extends through the carrier 5 adjacent the well units 10 to provide access to the bottom of the well therethrough. In this regard, the open top of each well unit 10, i.e. opening 37 and window 38, facilitate aspiration or pipetting within the well unit. As best seen in FIGS. 4 and 5, in one embodiment, a longitudinal slot, channel, or opening 39 extends along a lateral portion of body 30. Opening 39 facilitates fluid overflow and permits perfusion circulation when carrier 5 is used in combination with a perfusion bioreactor as described in more detail below. As also can be seen in FIG. 4, a ledge 41 may be provided adjacent the distal end of body 30 to accommodate a screen to hold scaffold 20 in a longitudinal direction, entrap cells or minimize particulate flow. For example, as shown in FIG. 6, screen 50 may be positioned and/or molded adjacent ledge 41 to prevent movement of scaffold 20 in the proximal direction while permitting fluid flow therethrough.

Scaffold handling system 1 and carrier 5 of FIGS. 1 and 2 are configured and dimensioned to be used with a multi-well plate having a plurality of main chambers or wells to house or contain a cell culture or cell culture experiment. Multi-well plates are well known to those skilled in the art. Exemplary multi-well plates include the BD Falcon™ multi-well plates, available in 24-well plates and 96-well plates. In this regard, carrier 5 of the present embodiment is configured and dimensioned to be inserted into and/or mate with such a multi-well plate. In operation, carrier 5 may be placed across a single row of the multi-well plate with each of the well units 11, 12, 13, and 14, extending into a corresponding well of the multi-well plate so that biological experimentation may be conducted. Multiple carriers 5 may be placed over additional rows of the multi-well plate such that a scaffold may be held in each well of the multi-well plate. For example, for a 24-well plate, six carriers 5 may be utilized with the 24-well plate. Of course, one skilled in the art will appreciate that any number of arrays and configurations may be utilized such that the entire multi-well plate may include a cell adherent scaffold.

Sidewalls or flanges 16, 18 of carrier 5 extend distally from the lateral sides of carrier 5 and are configured and dimensioned to extend about the lateral outside of the multi-well plate to accurately mate carrier 5 with the 24-well plate. As best seen in FIG. 2, flanges 16 and 18 may have a chamfered edge 19 for easy repositioning with respect to the multi-well plate. In addition, as best seen in FIG. 3, one or more nubs, locating pins, or protrusions 40 may be provided on the underside of carrier 5 to facilitate the alignment of carrier 5 with the individual wells of a multi-well plate. In this regard, the combination of protrusions 40, flanges 16, 18, and the geometry of carrier 5 lead to a reliable and repeatable system to hold scaffolds in place with respect to a multi-well plate.

In yet another embodiment, scaffold handling system 1 and carrier 5 of FIGS. 1 and 2 may also be used with a multi-well plate of a perfusion bioreactor, such as the perfusion bioreactors disclosed in U.S. Provisional Patent Application Ser. No. 60/699,849 entitled “Perfusion Bioreactor for Culturing Cells” filed Jul. 8, 2005, and PCT publication WO2006/033935, entitled “Perfusion Bioreactor for Culturing Cells,” (claiming priority to the aforementioned provisional application) filed Sep. 16, 2005, the entire contents of which are incorporated by reference. In this regard, carrier 5 of the present embodiment is configured and dimensioned to be inserted into and/or mate with such a multi-well plate of a perfusion bioreactor. In operation, carrier 5 may be placed across a single row of the multi-well plate of the perfusion bioreactor in the same manner as described above with respect to a 24-well plate with each of the well units 11, 12, 13, and 14, extending into a corresponding well of the multi-well plate of the bioreactor so that biological experimentation may be conducted. In this regard, the configuration and design of handling system 1 is advantageously configured to permit perfusion of cell culture media through the scaffolds. For example, the reliable and repeatable positioning of the carrier 5 is configured to hold the scaffold(s) 20 in the flow line of the perfusion bioreactor such that cell culture media flows through the scaffold from the distal end to the proximal end of each well unit 10. Overflow channel or opening 39 facilitates the return flow of perfusion media out though the proximal side of the scaffold 20.

Referring again to FIG. 1, an exemplary method of handling or manipulating a scaffold or scaffolds 20 according to the present invention is also shown. As shown with respect to well unit 11, as an initial step, a scaffold 20 or multiple scaffolds may be loaded or inserted into well units 10 of carrier 5. Once installed or loaded into carrier 5, as shown with respect to well unit 12, the scaffold(s) 20 may then be manipulated such as by being treated with chemicals, sterilized with ultraviolet radiation, seeded with cells, or other treatments. Similarly, as shown with respect to well unit 13, the scaffold may be inserted into a multi-well plate with cell culture media or biological agents to conduct biological experiments. As shown with respect to well unit 14 of FIG. 1, media can be perfused through scaffold(s) 20. Also, if microscopy is necessary, carrier 5 can be easily moved to a separate or fresh dry plate for microscopy without the need to handle the scaffolds directly.

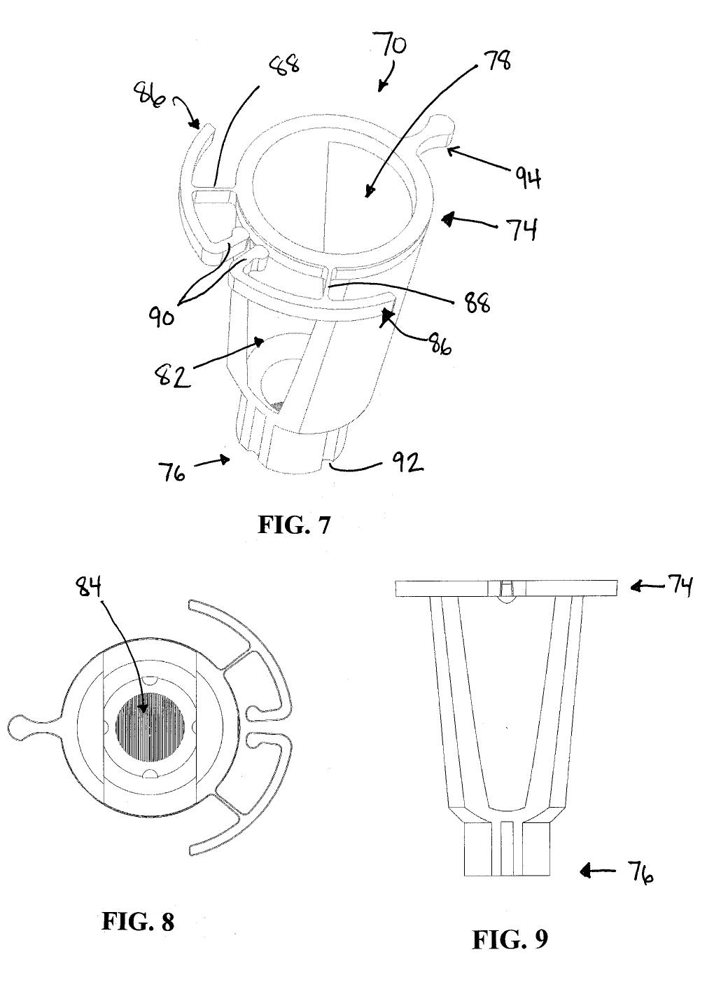

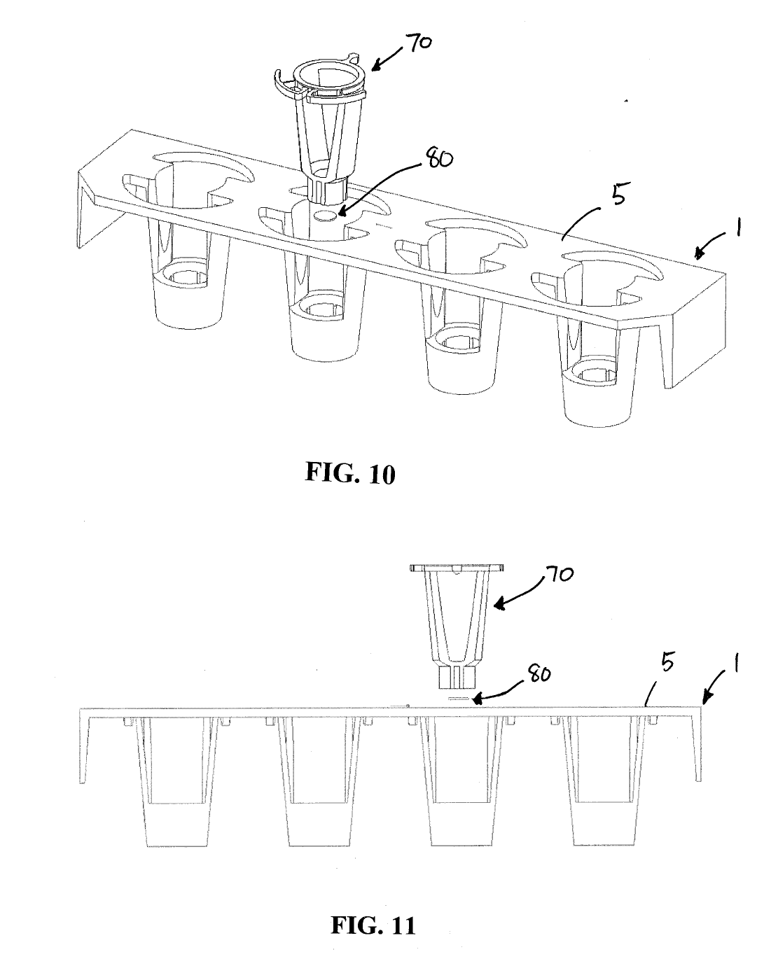

Turning now to FIGS. 7-11, an exemplary embodiment of a well insert or adapter 70 is shown. In one application, adapter 70 is particularly well-suited for housing, holding, handling, transporting or transferring a fragile or thin scaffold or sample, such as a tissue slice. As shown in FIGS. 10-11, adapter 70 is configured and dimensioned to be received within a well unit of scaffold handling system 1. Accordingly, adapter 70 has a similar size and shape to an individual well unit of system 1. Referring to FIG. 7, adapter 70 generally comprises a frustoconical or tapered body 72 extending from a top or proximal end 74 to a bottom or distal end 76. As best seen in FIG. 9, body 72 narrows from proximal end 74 to distal end 76. The size and dimension of tapered body 72 is configured to allow a single adapter 70 to be inserted into and retained within a well unit of system 1 to perform a biological experiment. Similar to well units 10, described above, the top or proximal end 74 comprises an opening 78 to permit physical and visual access to a sample 80 held therein. An opening 82 extends laterally through body 72 for aligning with opening 39 described above to allow for fluid overflow and perfusion circulation when carrier 5 is used in combination with a perfusion bioreactor. In this regard, as shown in bottom perspective view FIG. 8, a fluid permeable screen 84 may be provided and/or molded adjacent distal end 76 of adapter 70 for trapping, holding, or otherwise maintaining a tissue slice between adapter 70 and carrier 5 when the adaptor is inserted into a well unit of carrier 5.

Referring again to FIG. 7, adapter 70 may comprise mating or alignment features to facilitate engagement, alignment, and insertion of adapter 70 into a well unit of carrier 5. For example, according to one embodiment, adapter 70 may have fixturing nubs 86 to releasably engage carrier 5 and fix or lock adapter 70 in place within a well unit. Nubs 86 are interconnected to the outside of body 72 by radially extending living hinge members 88. Each nub 86 generally comprises tab portion 90 projecting radially inward from one lateral end to facilitate movement of nubs 86. For example, according to one application, tabs 90 may be pinched together such as by a tweezer, causing living hinges 88 to bend and allow movement of the nubs in the angular direction such that adapter 70 may be inserted into a well unit of carrier 5. Releasing the compressive or pinching force on the tabs will release nubs 86 to allow the nubs to engage, lock, or otherwise fix adapter 70 in place with respect to carrier 5. One skilled in the art will appreciate that such a feature helps to prevent accidental removal or dislodging of adapter 70 and the associated sample or tissue slice held therein. In another variation, adapter 70 may comprise slots or indentations 92 adjacent the distal end 76 to facilitate alignment of adapter 70 during insertion. According to another embodiment, adapter 70 may have a handle portion 94 extending radially outward from proximal end 74 of body 72 to facilitate handling of the adapter.

While the invention has been described in conjunction with specific embodiments and examples thereof, it is evident that many alternatives, modifications and variations will be apparent to those skilled in the art upon reading the present disclosure. Accordingly, it is intended to embrace all such alternatives, modifications and variations that fall within the spirit and broad scope of the appended claims.

What is claimed is:

1. A scaffold carrier cartridge system, comprising:
a carrier body including an array of well units, wherein a cell adherent scaffold may be positioned in each well unit.

2. The system of claim 1, wherein each well unit comprises a frustoconical body portion extending from the carrier body at a proximal end and includes a scaffold holding chamber at a distal end.

3. The system of claim 2, further comprising a scaffold housed within the scaffold holding chamber.

4. The system of claim 2, wherein scaffold holding chamber is tapered.

5. The system of claim 3, wherein the scaffold is releasably attached to the scaffold holding chamber by friction fit.

6. The system of claim 2, wherein at least one protrusion extends radially inward from a perimeter of the scaffold holding chamber to hold a scaffold therein by friction.

7. The system of claim 2, wherein the scaffold holding chamber may accommodate scaffolds with diameters ranging from about 4.8 mm to about 5.1 mm.

8. The system of claim 1, wherein the carrier body comprises four well units.

9. The system of claim 1, wherein the carrier body comprises eight well units.

10. The system of claim 1, wherein the carrier body comprises one well unit.

11. The system of claim 1, wherein the carrier body comprises a cross-member and a pair of flanges extending from the cross-member to engage with a multi-well plate.

12. The system of claim 1, wherein an opening is defined at a proximal end of each well unit to permit access to a scaffold held within the well unit.

13. The system of claim 1, wherein the carrier body further defines a window extending through the carrier body adjacent each well unit to provide access to the bottom of the well unit therethrough.

14. The system of claim 1, wherein the body of each well unit includes a channel to permit perfusion flow therethrough.

15. The system of claim 2, wherein the well unit body comprises an internal ledge to accommodate a screen to hold the scaffold in a longitudinal direction.

16. The system of claim 15, further comprising a screen positioned adjacent the ledge to prevent movement of the scaffold in the proximal direction while permitting fluid flow therethrough.

17. A bioreactor system, comprising the system of claim 1 in combination with a multi-well plate having a plurality of cell wells configured to contain cell cultures.

18. The system of claim 17, wherein a cell adherent scaffold may be positioned in each well unit, and wherein the cartridge system is coupleable with the multi-well plate such that each well unit aligns with a cell well of the multi-well plate.

19. A scaffold carrier insert, comprising:
a carrier insert body configured and dimensioned to be received within an individual well unit of the system of claim 1, wherein a cell adherent scaffold or tissue sample may be positioned in the carrier insert body.

20. The insert of claim 19, wherein the carrier insert body has a frustoconical or tapered shape extending from a proximal end to a distal end.

21. The insert of claim 20, wherein the proximal end comprises an opening for physical and visual access to a sample held therein.

22. The insert of claim 19, further comprising an opening extending laterally through the insert body for aligning with an opening of a well unit when the insert is positioned within the well unit.

23. The insert of claim 19, wherein a fluid permeable screen is provided adjacent distal end for contacting a tissue sample.

24. The insert of claim 19, wherein fixturing nubs extend outwardly from the insert body to releasably engage the carrier body.

25. The insert of claim 24, wherein the nubs are interconnected to the outside of the insert body by living hinge members and the nubs are moveable between first and second angular positions.

26. A scaffold carrier insert, comprising:
a carrier insert body configured and dimensioned to be received within an individual well unit of a scaffold handling cartridge including an array of well units, wherein a cell adherent scaffold or tissue sample may be positioned in the carrier insert body.

27. The insert of claim 26, wherein the carrier insert body has a frustoconical or tapered shape extending from a proximal end to a distal end.

28. The insert of clam 27, wherein the proximal end comprises an opening for physical and visual access to a sample held therein.

29. The insert of claim 26, further comprising an opening extending laterally through the insert body for aligning with an opening of a well unit when the insert is positioned within the well unit.

30. The insert of claim 26, wherein a fluid permeable screen is provided for contacting a tissue sample.

31. The insert of claim 26, wherein fixturing nubs extend outwardly from the insert body to releasably engage a portion of a scaffold handling cartridge.

32. The insert of claim 31, wherein the nubs are interconnected to the outside of the insert body by living hinge members and the nubs are moveable between first and second angular positions.
